(19) United States
(12) Patent Application Publication
(10) Pub. No.: US 2026/0295114 A1
(43) Pub. Date: Oct. 1, 2026

(54) SKIN TISSUE SUTURING COMPOSITION FOR WOUND HEALING AND REGENERATION, AND PREPARATION METHOD THEREFOR

(71) Applicants: DAEBONG LS CO., LTD., Incheon (KR); RESEARCH & BUSINESS FOUNDATION SUNGKYUNKWAN UNIVERSITY, Suwon-si, Gyeonggi-do (KR)

(72) Inventors: Seul Gi KIM, Suwon-si, Gyeonggi-do (KR); Jin Woong KIM, Seongnam-si, Gyeonggi-do (KR); Eun Ju PARK, Hwaseong-si, Gyeonggi-do (KR); Jin Oh PARK, Seoul (KR); Seon Mi LEE, Incheon (KR)

(21) Appl. No.: 19/479,518

(22) PCT Filed: Nov. 29, 2023
(86) PCT No.: PCT/KR2023/019454

§ 371 Date: Oct. 28, 2025

(30) Foreign Application Priority Data
Apr. 28, 2023 (KR) 10-2023-0056565

(51) Int. Cl. A61L 24/08; A61L 24/00

(52) U.S. Cl. CPC A61L 24/08; A61L 24/0031

(57) ABSTRACT

The present invention relates to a skin tissue suturing composition for wound healing and regeneration as a tissue adhesive system for skin tissue wound suturing and tissue regeneration that exhibits enhanced adhesion and cohesion by incorporating natural cellulose nanofibers, and a preparation method therefor. The composition is characterized by including an Al-BA complex compound where phenylboronic acid is conjugated with alginate, and bacterial cellulose nanofibers (BCNF). The invention enables effective wound suturing and rapid tissue regeneration simultaneously, with high biocompatibility and excellent physical and adhesive properties, and thus is suitable for use not only in soft tissues but also as a universal alternative to traditional sutures or staples. Furthermore, with ability to be used for suturing various tissue types beyond the skin, the composition is expected to find active applications in various regions including medical and pharmaceutical fields.

### TECHNICAL FIELD

The present invention relates to a skin tissue suturing composition for wound healing and skin tissue regeneration as a tissue adhesive system for skin wound suturing and tissue regeneration having improved adhesiveness and cohesion using natural cellulose nanofibers, and a method of preparing the same.

### BACKGROUND ART

In modern society, wounds frequently occur due to excessive UV exposure, trauma, and long-term, repeated skin infections. Exposure to various harmful substances at these sites may also lead to skin diseases. To prevent these problems, there is a need for a system that reduces the risk of infection by simultaneously achieving rapid tissue suturing and regenerative performance at the wound site.

Currently, sutures or staples are generally used for wound suturing. However, this requires additional removal after the procedure. Furthermore, the applicability thereof depends on the wound site, size, and depth, in addition to the risk of infection. This limits the applicability to micro-wounds. For this reason, there is significant interest in the development of medical adhesives that can reduce the risk of external infection by providing strong adhesion and rapid wound suturing. The most widely used tissue adhesives are cyanoacrylates. Cyanoacrylates are widely used due to the excellent adhesive properties thereof, but have limitations in that they readily flow due to low viscosity and are brittle after curing due to low elongation. Furthermore, it is difficult to apply cyanoacrylates to organs other than the skin tissue due to poor biocompatibility thereof.

In order to solve the low elongation of cyanoacrylate-based topical skin adhesives, silicone-based topical skin adhesives have been proposed (silicone-based adhesives can elongate to 160% of the original length and fully return to the original dimensions thereof when use in combination with conventional wound suturing devices). However, there is need for great improvement of curing speed and biocompatibility.

The prior art could be understood more specifically from Korean Patent Publication No. 10-2023-0019455 (Patent Document 1), or the like. The entire content of Patent Document 1 is hereby incorporated by reference.

### PRIOR ART DOCUMENT

### Patent Document

(Patent Document 1) KR 10-2023-0019455 A1 (Feb. 8, 2023)

### DISCLOSURE

### Technical Problem

Recently, the development of natural tissue adhesives with enhanced biocompatibility is actively underway. These adhesives typically utilize fibrin proteins, gelatin, and polysaccharide polymers such as alginate and chitosan based on the hemostatic mechanisms of the human body. These adhesives can bond tissues due to excellent biostability without inducing immune responses or inflammation, but have limited general application because they are readily removed by external forces due to weak inherent physical properties and adhesive strength. In particular, conventional tissue adhesive systems are applicable only to soft tissue due to weak physical properties and adhesive strength, and require introduction of additional drugs in order to induce tissue regeneration.

In order to solve these problems, attempts are being made to improve the physical properties and adhesive strength of natural tissue adhesives by adding crosslinking agents, nanoparticles and the like. However, cyanoacrylates and natural tissue adhesives only have wound suturing performance and have no other efficacy.

Therefore, it is an object of the present invention to provide a highly biocompatible natural tissue adhesive that simultaneously provides effective wound suturing and rapid tissue regeneration.

### Technical Solution

In order to solve the problems of the prior art, the present inventors prepared an adhesive hydrogel for skin wound suturing by improving the physical properties of the adhesive system and adhesion to tissue, and introducing natural cellulose having a structure similar to the extracellular matrix and verified the effects of natural cellulose on improving adhesive performance and promoting skin wound regeneration.

In accordance with the present invention, the above and other objects can be accomplished by the provision of a suturing composition containing an Al-BA complex compound in which phenylboronic acid is conjugated to alginate, and bacterial cellulose nanofibers (BCNF).

In an embodiment, a weight ratio of the Al-BA complex compound to the bacterial cellulose nanofibers (BCNF) may be 1:0.01 to 1:0.5.

In an embodiment, the Al-BA complex compound may be present in an amount of 1 to 10 wt % with respect to the total weight of the composition.

In an embodiment, the bacterial cellulose nanofibers (BCNF) may be present in an amount of 1 wt % or less with respect to the total weight of the composition.

In an embodiment, the bacterial cellulose nanofibers (BCNF) may be positively charged.

In an embodiment, the composition may be provided in the form of an adhesive hydrogel.

In an embodiment, the composition may be applied to skin tissue sutures for wound healing and regeneration.

In accordance with another aspect of the present invention, there is provided a suturing device impregnated with or having at least one surface coated with the suturing composition of the present invention.

In accordance with another aspect of the present invention, there is provided a method of preparing a suturing composition, the method including: conjugating 3-aminophenylboronic acid to an alginate polymer to prepare an Al-BA complex compound;
positively charging bacterial cellulose nanofibers (BCNF) to obtain (+) BCNF; and
mixing the Al-BA complex compound with the (+) BCNF to prepare an adhesive hydrogel.

### Advantageous Effects

The suturing composition according to the present invention enables both effective wound suturing and rapid tissue regeneration, and exhibits high biocompatibility and superior physical and adhesive properties, thus being suitable for general use in addition to application to soft tissues and being a viable alternative to conventional sutures or staples. Furthermore, the present invention is applicable to various tissues in addition to skin, which supports that the suturing composition has the potential for wide application to various fields including the medical and pharmaceutical fields.

### DESCRIPTION OF DRAWINGS

FIG. 1 shows (a) a schematic diagram illustrating alginate-boronic acid (Al-BA) conjugation, (b) FT-IR analysis of Al-BA synthesis, and (c) quantification of conjugated boronic acid.

FIG. 2 shows (a) a schematic diagram illustrating the synthesis of positively charged natural cellulose ((+)BCNF), (b) surface potential of natural cellulose depending on the amount of glycidyltrimethylamine chloride (GTAC) added, and (c) a TEM image of (+)BCNF.

FIG. 3 shows (a) a sol-gel transition image of Al-BA/(+)BCNF hydrogels at various PH levels, (b, c) rheological analysis, (d) a schematic diagram illustrating the durability test of Al-BA/(+)BCNF hydrogels, and (e) quantification of the maximum rupture pressure depending on the (+)BCNF content.

FIG. 4 shows (a) an image and schematic diagram illustrating the adhesion test of the Al-BA/(+)BCNF hydrogel, (b) quantification of adhesion depending on the (+)BCNF content, (c) quantification of adhesion depending on the content of GTAC introduced onto the natural cellulose surface, and (d,e) images of the ex vivo adhesion performance of the Al-BA/(+)BCNF hydrogel.

FIG. 5 shows (a) a schematic diagram illustrating the in vitro wound healing performance test of the adhesive hydrogel, (b,c) wound healing performance test of keratinocytes on the adhesive hydrogel, and (d,e) wound healing performance test of fibroblasts on the adhesive hydrogel.

FIG. 6 is a schematic diagram illustrating a process of skin wound suturing and healing using the BCNF-based pH-sensitive injectable tissue adhesive.

### BEST MODE

Hereinafter, the present invention will be described in detail.

The present invention relates to a tissue adhesive system for skin wound suturing and tissue regeneration with enhanced adhesive and cohesive properties using natural cellulose nanofibers. Conventional natural tissue adhesives based on cyanoacrylate and polysaccharide polymers have been used for wound suturing, but have limitation on effective wound suturing performance due to low biocompatibility and adhesive force. Therefore, in accordance with the present invention, natural cellulose nanofibers which exhibit strong physical properties and have a structure similar to the extracellular matrix are introduced into an alginate-based tissue adhesive capable of sol-gel transition in a biological pH environment, to prepare an adhesive hydrogel and a tissue suturing system with improved adhesive performance with skin tissue and rapid tissue regeneration at the wound site is developed based thereon. Specifically, the present invention aims to establish a process of preparing a pH-sensitive injectable tissue adhesive system using natural cellulose nanofibers in order to construct an adhesive system for effective wound suturing and tissue regeneration.

In order to achieve this, the present invention is directed to a suturing composition containing an Al-BA complex compound in which phenylboronic acid is conjugated to alginate, and bacterial cellulose nanofibers (BCNF). Al-BA having a configuration in which alginate is conjugated with 3-phenylboronic acid may induce tissue adhesion through rapid sol-gel transition in a biological pH environment. To enhance the physical properties of the hydrogel and the affinity of the hydrogel for tissues, positively charged natural cellulose nanofibers ((+)BCNF) are further introduced into Al-BA to prepare an adhesive hydrogel. The cellulose nanofibers not only enhances the physical properties and adhesive performance of the adhesive, but also contributes to accelerated tissue regeneration.

In particular, in an embodiment, a weight ratio of the Al-BA complex compound to the bacterial cellulose nanofibers (BCNF) may be 1:0.01 to 1:0.5, preferably, 1:0.02 to 1:0.2, and more preferably, 1:0.03 to 1:0.16. This is because excellent physical properties can be obtained within this weight ratio range and better understanding thereof is possible from the examples and experimental examples described below.

In an embodiment, the Al-BA complex compound may be present in an amount of 1 to 10 wt %, preferably, 2 to 5 wt %, with respect to the total weight of the composition.

In an embodiment, the bacterial cellulose nanofibers (BCNF) may be present in an amount of 1 wt % or less, preferably, 0.01 to 0.5 wt %, with respect to the total weight of the composition.

In an embodiment, the suturing composition may be provided in the form of an adhesive hydrogel. Preferably, the suturing composition may be applied to skin tissue sutures for wound healing and skin regeneration and may be applied to sutures of tissues other than skin.

Furthermore, the composition of the present invention may be used alone or in combination with a wound suturing device. The wound suturing device suitable for use in the present invention include any device configured to close the wound. Non-limiting examples of useful wound suturing devices include wound suturing strips, tapes, patches, or any other material suitable for closing a wound, preferably, strips.

Furthermore, in one embodiment of the wound suturing device, the suturing device may be porous, allowing a flowable polymeric adhesive to penetrate the device so that it can be properly bonded to the tissue surface. As used herein, the term “porous” means that the wound suturing device has a majority of pores such that a subsequently applied polymeric adhesive composition is sucked in or absorbed by a bulk material, or that the wound suturing device has a majority of pores (such as a net or screen) such that a subsequently applied polymeric adhesive composition passes directly through the bulk material while the composition is sucked in or absorbed by the bulk material. For example, in textile materials, the term “porous” is generally used to mean that the applied adhesive composition penetrates and passes through the pores between the fibers, but does not necessarily penetrate and pass through the fibers. In this regard, a preferred wound suturing device may be a mesh strip.

Furthermore, in another embodiment, the wound suturing device may include a wound-facing surface and a top surface, and the wound-facing surface contains the suturing composition of the present invention applied over at least a portion thereof. Furthermore, in addition to the suturing composition of the present invention, the wound suturing device may further contain other topical skin adhesives (TSA) or post-surgery adhesives (PSA).

The suturing composition according to the present invention may be prepared through the following steps.

First, in accordance with the present invention, a pH-sensitive injectable adhesive hydrogel containing natural cellulose for wound suturing and rapid tissue regeneration is prepared. Specifically, an alginate-boronic acid adhesive capable of sol-gel transition in a biological pH environment is prepared.

Then, positively charged natural cellulose nanofibers are further introduced to enhance physical properties and promote tissue regeneration, to ultimately establish an adhesive hydrogel system. The adhesive hydrogel system improves the physical properties and adhesive performance of the hydrogel and accelerates the proliferation of skin cells at the wound site due to the structure thereof similar to the extracellular matrix, to establish a system advantageous for wound suturing and healing.

Specific details of the method are as follows. (1) introducing 3-aminophenylboronic acid into alginate to prepare a pH-sensitive adhesive, (2) preparing positively charged natural cellulose nanofibers to improve adhesion to tissue and regeneration performance, and (3) mixing the two materials to prepare an adhesive hydrogel.

Hereinafter, the present invention will be described in more detail using examples and experimental examples. However, these examples and experimental examples are provided only for better understanding of the Detailed Description of the present invention and should be not construed as limiting the scope of the present invention.

### Mode for Invention

### EXAMPLE

Preparation of Al-BA complex compound

An alginate polymer was dispersed in MES buffer (0.1 M, pH 5.0) at room temperature. A mixed solution of ethyl-3-(3-dimethylaminopropyl) carbodiimide (EDC)/N-hydroxysuccinimide (NHS) was slowly added to the alginate dispersion while stirring. A 3-aminophenylboronic acid solution was then slowly added and stirred for 24 hours. After the reaction was completed, the residue was removed through dialysis to prepare alginate-boronic acid (Al-BA). The prepared Al-BA was lyophilized and stored.

### Preparation of (+)BCNF

A natural cellulose raw material was dispersed in a 0.1M NaOH solution, a predetermined amount of glycidyltrimethylamine chloride was added thereto, and the mixture was stirred at 60° C. for 2 hours. Then, the residue was removed by centrifugation and the reaction product was redispersed in a 0.01 M NaOH solution. NH₄OH was added to the cellulose redispersion, followed by stirring at 60° C. for 2 hours, and centrifugation to obtain positively charged natural cellulose nanofibers ((+)BCNF).

### Preparation of Adhesive Hydrogel

(+)BCNF was added to 3 wt % Al-BA at concentrations of 0.1 (Example 1), 0.25 (Example 2), and 0.5 (Example 3) wt % to prepare adhesive hydrogels, as shown in Table 1 below, and the physical properties and adhesive performance of adhesive hydrogels were tested in experimental examples.

### Comparative Example

In order to measure the physical properties and adhesive performance of the adhesive hydrogels depending on the amount of (+)BCNF added, an Al-BA sample containing no (+)BCNF was used as a control group, as shown in Table 1 below.

TABLE 1

| | Al-BA | (+) BCNF concentration |
|---|---|---|
| | concentration (wt %) | (wt %) |
| Comparative Example | 3 | 0 |
| Example 1 | 3 | 0.1 |
| Example 2 | 3 | 0.25 |
| Example 3 | 3 | 0.5 |

### Experimental Example

### Experimental Example 1

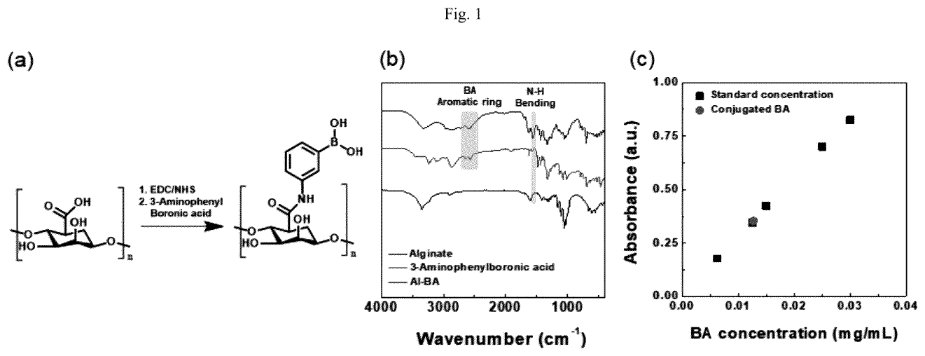

FT-IR analysis was performed before and after the addition of 3-aminophenylboronic acid to the alginate polymer. Similar peaks were observed at 3,000 to 2,800 cm⁻¹ and 1,650 to 1,580 cm⁻¹, and whether or not synthesis occurred was determined. The 3-aminophenylboronic acid was quantified using UV-Vis analysis. The results are shown in FIG. 1.

### Experimental Example 2

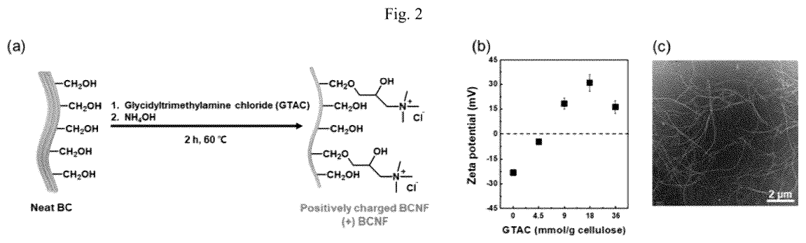

The surface potential of natural cellulose nanofibers was analyzed depending on the amount of glycidyltrimethylamine chloride (GTAC) functional groups introduced. As the amount of GTAC introduced increased, the surface potential increased in a positive range. Furthermore, the nanofiber structure of the prepared (+)BCNF was visualized using TEM imaging. The results are shown in FIG. 2.

### Experimental Example 3

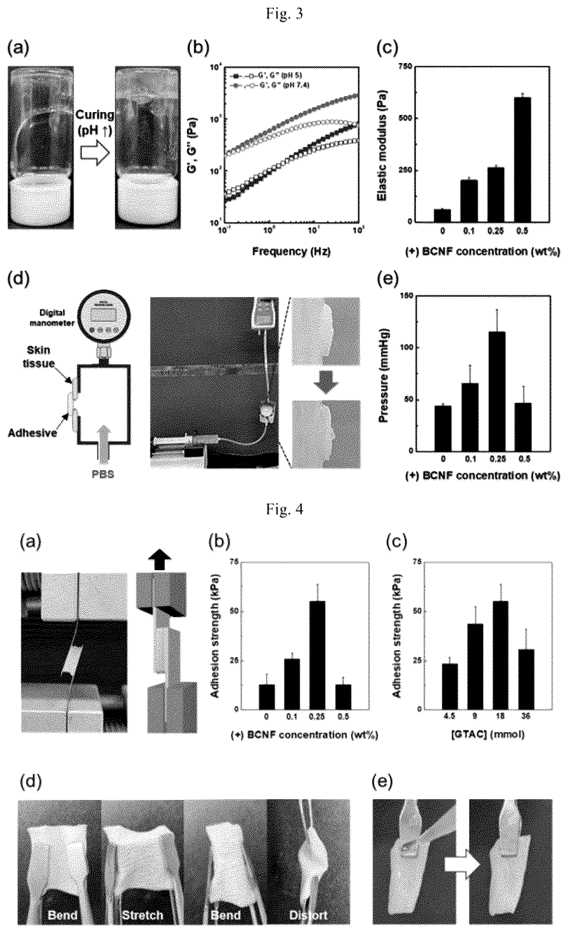

A solution was prepared by mixing the Al-BA with (+)BCNF and whether or not a sol-gel transition occurred as the pH increased was determined by rheological analysis. The physical properties of the formed adhesive hydrogel were quantified depending on the amount of (+)BCNF introduced. The results showed that the physical properties increased linearly with an increasing amount of (+)BCNF. In addition, the durability of the hydrogel in tissue was tested through a rupture test. The resistance of hydrogel to pressure in the container while adding PBS. The result showed that the resistance to pressure increased with an increasing concentration of (+)BCNF, but decreased at 0.5 wt %. These results are shown in FIG. 3. However, this behavior is also related to tissue adhesion and thus a comparative analysis was conducted in Experimental Example 4, as described below.

### Experimental Example 4

The adhesive hydrogel was applied to a pig skin with a predetermined size, Tris buffer (pH 8.0) was added dropwise and cured for 1 hour. The adhesive strength was quantified using a universal testing machine (UTM). The results showed that the introduction of (+)BCNF improved the adhesive performance, and the adhesive performance at various concentrations exhibited similar behaviors, like the rupture test. It was determined that the adhesive hydrogel at a predetermined concentration had an uneven polymer dispersion due to the difference in viscosity between Al-BA and (+)BCNF and thus actually hindered the adhesive performance. Therefore, the adhesive hydrogel containing 0.25 wt % (+)BCNF was selected as the optimal concentration for excellent performance. The details and results of Experimental Example 4 are shown in FIG. 4.

### Experimental Example 5

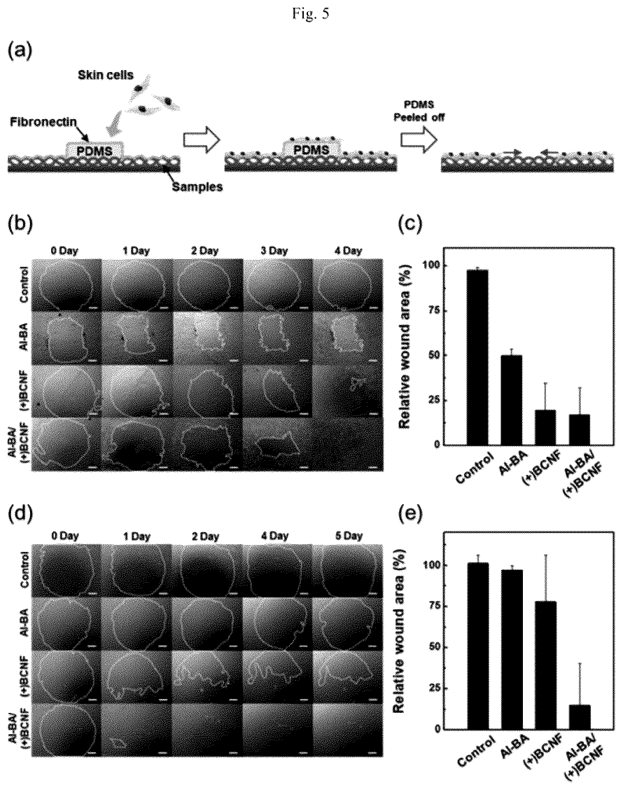

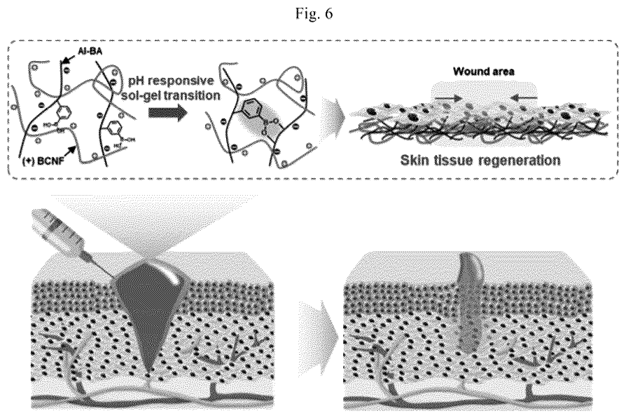

The wound healing performance of the adhesive hydrogel was tested using fibroblasts and keratinocytes as representative skin cells. A substrate was coated with a sample of each ingredient and cells were cultured. Changes in wound size over time were observed based on the substrate. The results showed that (+)BCNF having a structure similar to the extracellular matrix facilitated cell proliferation at the wound site and resulted in effective wound healing. These results are shown in FIG. 5.

### INDUSTRIAL APPLICABILITY

The present invention is applicable to industries such as medical, pharmaceutical, and nursing.

1. A suturing composition comprising:
an Al-BA complex compound in which phenylboronic acid is conjugated to alginate; and
bacterial cellulose nanofibers (BCNF).

2. The suturing composition according to claim 1, wherein a weight ratio of the Al-BA complex compound to the bacterial cellulose nanofibers (BCNF) is 1:0.01 to 1:0.5.

3. The suturing composition according to claim 1, wherein the Al-BA complex compound is present in an amount of 1 to 10 wt % with respect to a total weight of the composition.

4. The suturing composition according to claim 1, wherein the bacterial cellulose nanofibers (BCNF) are present in an amount of 1 wt % or less with respect to the total weight of the composition.

5. The suturing composition according to claim 1, wherein the bacterial cellulose nanofibers (BCNF) are positively charged.

6. The suturing composition according to claim 1, wherein the composition is provided in the form of an adhesive hydrogel.

7. The suturing composition according to claim 1, wherein the composition is applied to skin tissue sutures for wound healing and regeneration.

8. A suturing device impregnated with or having at least one surface coated with the suturing composition according to claim 1.

9. A method of preparing a suturing composition, the method comprising:
conjugating 3-aminophenylboronic acid to an alginate polymer to prepare an Al-BA complex compound;
positively charging bacterial cellulose nanofibers (BCNF) to obtain (+)BCNF; and
mixing the Al-BA complex compound with the (+)BCNF to prepare an adhesive hydrogel.
